(19) United States
(12) Patent Application Publication
(10) Pub. No.: US 2010/0204761 A1
(43) Pub. Date: Aug. 12, 2010

(54) Intravenous laser/non-laser light emitting diode implant for destroying blood borne viral infestations and other malign cells, integrated among blood components in a human circulatory system

(76) Inventor: John K. Murray, Tenn.Colony, TX (US)

Correspondence Address: Patricia Black, 2307 Monte Carlo #B, Arlington, TX, 76015, US

(21) Appl. No.: 12/378,053

(22) Filed: Feb. 11, 2009

(51) Int. Cl. A61N 5/067

(52) U.S. Cl. 607/89

(57) ABSTRACT

Intravenous laser and non-laser light-emitting diode (LED) implant having the capacity to be fitted with a plurality of numerous separate nanometers of laser or non-laser light, attached radially in an inwardly-facing, saline-filled inflatable ring where LEDs are ran in sequence on separate contact tracks that are attached to a dual-strand cord, which leads to a component containment compartment housing certain electronic components, remote signal receiver, and a battery. A preferred embodiment includes the inflatable ring of LEDs, surgically inserted into a human vein by-way of catheter, inflated by syringe with a saline solution, to provide irradiation of cancerous cells or viruses incorporated with circulated blood, activated by a hand-held remote control.

### STATEMENT OF FEDERALLY SPONSORED RESEARCH OR DEVELOPMENT

Not Applicable

### DESCRIPTION OF APPENDIX

Not Applicable

### BACKGROUND OF THE INVENTION

This invention relates generally to the field of implantable medical devices, and more specifically to an intravenous, light-radiating implant, activated by a remote control.

Numerous of established medically and scientifically sound applications of specific nanometers of visible and invisible laser and non-laser light have been utilized in the process of destroying cancerous human cells, and an assortment of viral and bacterial infestations.

Some of the applications of light were incorporated with light-sensitizing dyes or contrast agents introduced into targeted malign cells, rendering these cells vulnerable to subjection to exposure to collimated beams of light. When these light-absorbent chemicals are struck by these beams, the chemicals break apart into substances that destroy the targeted cells. Another application utilizes a low-power pulsating laser that requires no sensitizing chemicals to create vulnerability, but rather utilizes visible violet light at the 425 nanometer range of the spectrum. This “excitation” laser creates a vibration significant to rupture the capsid or “shell” which envelopes viral DNA or RNA (genetic material), rendering the virus unable to enter a human lymphocite cell where it would ordinarily commandeer the lymphocite's genetic material to replicate itself. Pulsation of this collimated beam of violet light provides a cooling-off period which protects blood cells within close proximity to targeted viruses.

Yet another application of light involves the introduction of psoralens, taken orally, that enter the circulatory system to intermingle with blood components. Though otherwise innocuous, these microscopic compounds become active molecular surgeons that serve to chip away chemical links that bind the DNA molecules of the cells involved in lymphomatic leukemia and T-cell lymphoma, when blood is exposed to wider-range waves of invisible ultraviolet light.

In all cases of exposure to light to decrease the number of targeted cells, blood is pumped out of the patient's body through clear tubes where it then passes through specific nanometers of laser or non-laser light and is reintroduced back into the patient's body. There are, however, extremely detrimental side effects associated with the continuous routing of blood from and back into the body. Blood removed multiple times from its natural habitat begins to separate or disintegrate. This condition is called hemolysis and can even prove fatal at times. And because viral cells also tend to infest non-blood tissue, discontinuation of out-of-body routing would result in rapid reinfestation of viruses into the circulatory system. The need for a method and device to take the light to the blood in a practical and effective manner resulted in the process and invention herein.

### BRIEF SUMMARY OF THE INVENTION

The primary object of the invention is to provide a multiple-spectrum, laser or non-laser, non-pharmaceutical, intravenous implant that, through its application, destroys genetic material that is instrumental in the process of replication of certain viral cells.

Another object of the invention is to provide a means through which to remotely-activate a photodynamic process of destroying blood borne cancer cells, in a time-predictable manner, after the introduction of a contrast agent sensitizes the targeted malign cells.

Another object of the invention is to provide a means through which to expose human blood to specific nanometers of light without the necessity of removing the blood from the patient's body.

A further object of the invention is to provide a process through which the elimination of blood borne human immunodeficiency virus and numerous of other viral infestations may be affected without the detrimental side effects associated with many antiviral pharmaceuticals.

Other objects and advantages include the conformation of the circumference of the invention to accommodate the inner perimeter of the selected vein, on the inside of the upper-thigh. The invention is designed to be “adjustable”, reducing the number of LEDs in a larger model and utilizing a number sufficient for a smaller circle.

Still another object of the invention is to provide an in-vein light “filter” that would remain activated to constantly maintain viral load at an immeasurable level to afford a human body's own immune-response mechanism to produce antibodies sufficient to combat viral cells in non-blood tissue, using total-surface light-emitting diodes in the purple portion of the visible light spectrum in the form of a very low power femtosecond laser system.

Further objects and advantages of the present invention will become apparent from the following descriptions, taken in conjunction with accompanying drawings, wherein, by way of example and illustration, an embodiment of the present invention is disclosed.

In accordance with a preferred embodiment of the invention, there is disclosed Intravenous Laser/Non-Laser Light-Emitting Diode Implant For Destroying Blood Borne Viral Infestations And Other Malign Cells Integrated Among Blood Components In A Human Circulatory System comprising: a plurality of select laser or non-laser LEDs attached radially in an inwardly-facing manner to a flexible, Inflatable ring; a one-way valve; flexible two-track contact points; a power cord attached to either of the two tracks, and at the other end, a containment compartment in which are enclosed a battery, signal-receiver for activation, as well as other standard electronic components such as resistars and battery contacts.

The invention is to be implanted in a major vein, by-way of catheter, within a person afflicted with blood-associated cancer or viral infection to afford interaction of light, laser or non-laser, with psoralens or light sensitivity enhancing chemicals, or through energy-produced vibrations produced by a low-power violet excitation laser designed to pulsate every one hundred quadrillionths of a second, with laser density of 5 microjoules per square centimeter. This low density laser light is such that human cells within close proximity of viral cells would remain undamaged.

### BRIEF DESCRIPTION OF DRAWINGS

The drawings constitute a part of this specification and include exemplary embodiments to the invention, as well as examples of intravenous insertion. It should be understood that in some instances, various aspects of the invention, its components, and the processes of insertion may be miniaturized or enlarged to facilitate an understanding of the invention. Various subtle modifications may be required to accommodate the vein insertion in a child.

FIG. 1 Gives an inventory of entire required components to affect begin-to-end process.

FIG. 2 Shows a section view of the circular LED implant, air bleed-valve, and the circuit containment compartment and its contents.

FIG. 3 Shows all components, assembled, in cut-away form.

FIG. 4 Is the circular LED implant, connected to the closed containment compartment by a 2-strand cord.

FIG. 5 Shows flexible contact tracks and inside plastic layer with holes to accommodate LEDs, and the 2-strand cord attached to ends of the tracks.

FIG. 6 Shows contact tracks and an adhesive utilized to adhere tracks to the inner surface of the inside plastic layer, and liquid solder to affix the LEDs to each of the 2 tracks. Adhesive around the holes secure LEDs to inner circumference of hole.

FIG. 7 Shows fully assembled circuit of ten LEDs, and triple plastic layers heat-sealed at the bottom edges. Saline feed valve is in place.

FIG. 8 Shows all three layers of the LED circle, separate, and a view of all three, heat-sealed at their bottom edges.

FIG. 9 Shows the two layers of plastic designed to eventually contain saline, and the one-way saline injection valve, dismantled, and then assembled.

FIG. 10 Shows a heat sealing process of plastic layers.

FIG. 11 Is a view of the inflated circle of LEDs, from below.

FIG. 12 Is a partially sectioned view of the circle showing inner LEDs, track, saline valve and cord.

FIG. 13 Shows a sectioned view of saline containment area, LEDs, tracks, valve and cord.

FIG. 14 Expresses the concept of a circle of LEDs, radiating light radially.

FIG. 15 Shows flexible catheter and saline injector barrel.

FIG. 16 Shows threaded end of the injector barrel being attached to the syringe.

FIG. 17 Shows injector barrel's needle submerged in saline as plunger draws saline into the syringe.

FIG. 18 Shows the end of the saline feed barrel feeding up into the end of the catheter, and the cord being fed into the capsule end of the catheter.

FIG. 19 Shows deflated circle of LEDs being rolled into “package”, and the air bleed-valve designed to be inserted into valve to allow evacuation of entrapped air to afford smallest possible “package”.

FIG. 20 Shows tightly rolled “package” with saline-filled feed barrel ready for attachment to valve.

FIG. 21 Shows multiple views and section views of valve and threaded injector barrel, with hollow needle.

FIG. 22 Shows cross-section view of deflated LED circle, and injector barrel and needle in place for threaded attachment.

FIG. 23 Shows injector barrel attached to valve, readying “package” to be drawn down into the capsule by pulling on the syringe at the opposite end of the catheter.

FIG. 24 Is an aerial view of the LED “package”, fully submerged down into the capsule.

FIG. 25 Shows insertion of the capsule and of the catheter into the incision on the inner-thigh of the recipient, and on into the common iliac vein.

FIG. 26 Shows catheter positioned at desired location, sectioned to show content of catheter, with arrow to indicate direction of saline feed barrel when “package” is pushed from capsule to vein.

FIG. 27 Shows “package” exiting capsule and entering vein.

FIG. 28 Shows “package” completely freed from capsule and ready for inflation process.

FIG. 29 Shows ultrasound device and visual monitor utilized in ultrasonography to track insertion and inflation of LED circle.

FIG. 30 Shows section views of saline containment area and valve and injector barrel as plunger forces saline from the syringe and up into the circle of LEDs.

FIG. 31 Shows the LED circle as it fills with saline and fills the inner-perimeter of the vein.

FIG. 32 Shows entire process of saline injection/inflation of LED circle in the common iliac vein of the recipient.

FIG. 33 Shows fully inflated LED circle, burrs set in vein, and an arrow to indicate disconnection of injector barrel from valve by turning opposite end of barrel.

FIG. 34 Shows disconnected injector barrel as barrel and catheter now are pulled down and out of the vein.

FIG. 35 Shows injector barrel and catheter removed, leaving LED circle secured stationary by multiple burrs, and 2-strand cord leading from LED circle to vein and thigh incisions.

FIG. 36 Shows 2-strand cord protruding from vein, now sutured closed, and the ends of the cord separated and ends cleared of insulation.

FIG. 37 Shows cleared copper wire at the ends of the cord being secured by a screwdriver to the contact points inside the circuit containment compartment.

FIG. 38 Shows ends of cord fully attached to contact points making containment compartment ready for closing and sealing.

FIG. 39 Shows the closed and sealed circuit containment compartment being inserted beneath the skin of the inner-thigh of the recipient.

FIG. 40 Shows insertion process, in its completion, with section views of LED circle with arrow to indicate direction of circulated blood through the circle of LEDs, the implanted circuit containment compartment, sutured incision on inner-thigh, and remote-control which now activates the LEDs intended to affect irradiation of malign cells, integrated with blood components circulating through the radiated light.

FIG. 41 Is a schematic of a typical drive circuit designed to maintain a precise, non-fluxuating source of constant energy to safely power the circle of LEDs.

FIG. 42 Shows close-up view of circle of LEDs with an arrow to indicate direction of circulating blood.

FIG. 43 Shows a section view of the circle of LEDs, its saline containment area, and small burr that facilitates stationary stability.

FIG. 44 Is an aerial view of the circle of LEDs, with arrows indicating radial irradiation of light into circulating blood in a human blood vein.

### DETAILED DESCRIPTION OF THE PREFERRED EMBODIMENTS

Detailed descriptions of the preferred embodiments are provided herein. It should be understood, however, that the present invention may undergo changes in size and form to accommodate a smaller circumference or location of a selected vein. Specific details disclosed herein are not to be interpreted as limiting, but rather as a basis for the claims and as a representative basis for instructing one skilled in intravenous, catheter-deployed medical devices. (See FIGS. 45-48 for examples of alternative design.)

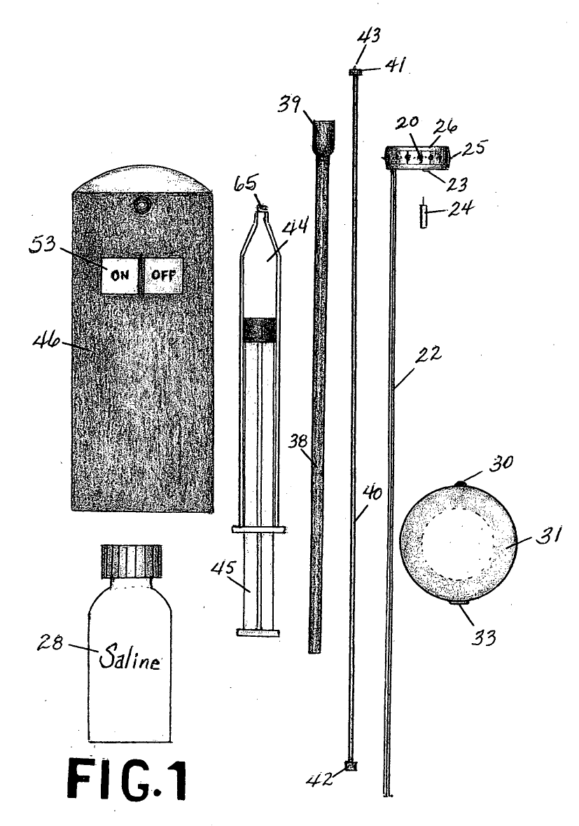

FIG. 1 shows a complete inventory of apparatus and necessities to affect the process of intravenous implantation of the invention 100. FIG. 4 shows circle 26 of LEDs 20, cord 22, and the circuit containment compartment 31, which are all that will remain implanted.

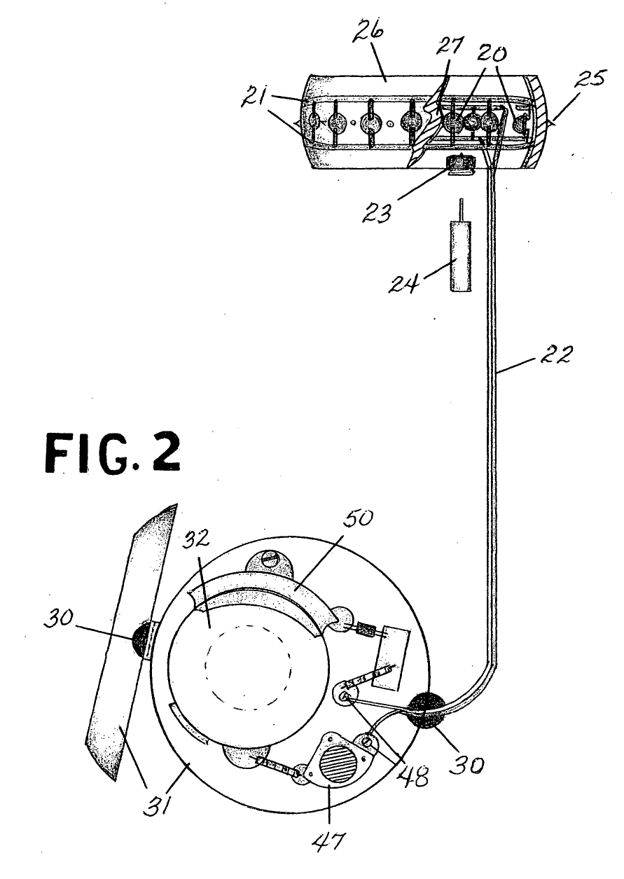

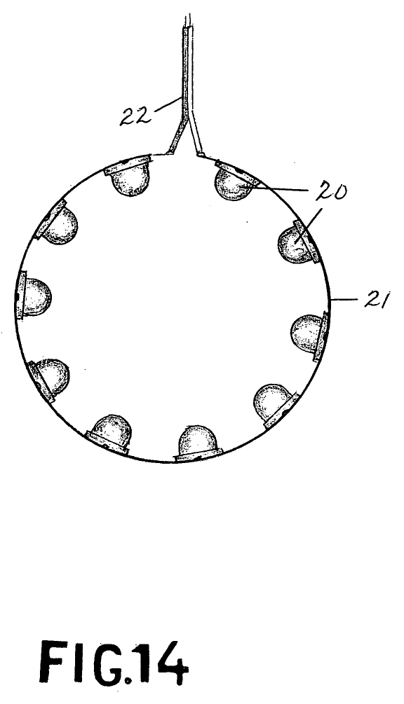

Invention's 100 saline 28 filled circle 26 of LEDs 20, as shown in sectioned view in FIG. 2, is comprised of two contact tracks 21, to which multiple of LEDs 20 are attached radially, as also shown in FIGS. 11 and 14, facing inwardly toward the center of the circle 26.

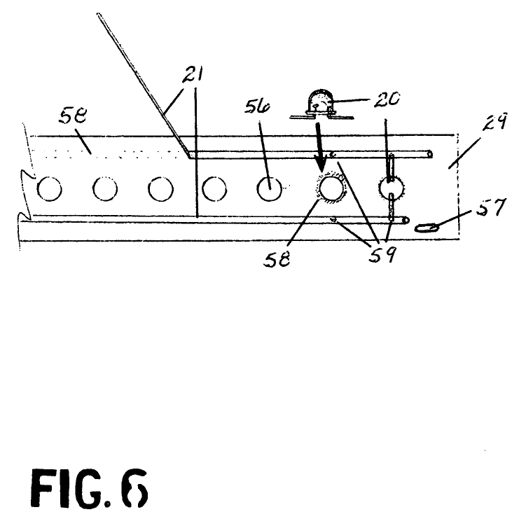

FIG. 6 shows the inner-sheet 29 of plastic to which the two flexible tracks 21 are affixed with an adhesive 58. LEDs 20 are then positioned into pre-cut holes 56, with adhesive 58, and affixed to the tracks 21 with a liquid solder 59. Tracks 21 are electrically conductant, thin, and flexible, as also indicated in FIG. 6.

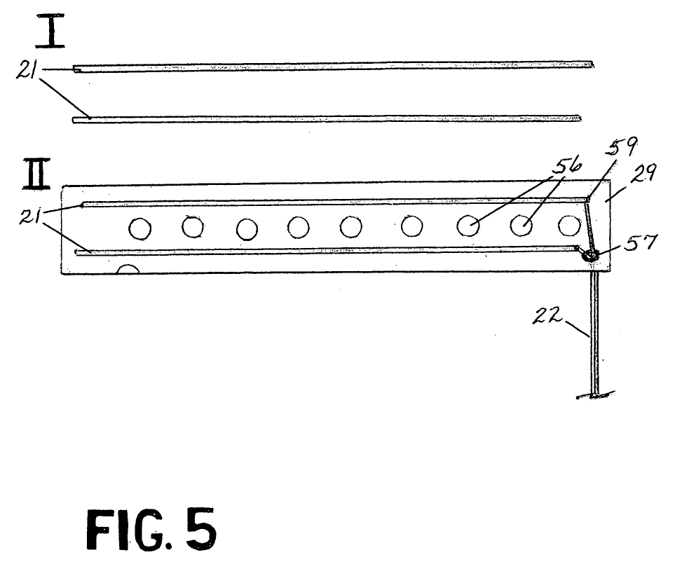

A two-strand cord 22 is connected with liquid solder 59 to the end of each track 21, then inserted into a small hole 57 where the cord 22 protrudes out the same side as the faces of the LEDs 20, as shown in FIGS. 5, 7 and 8.

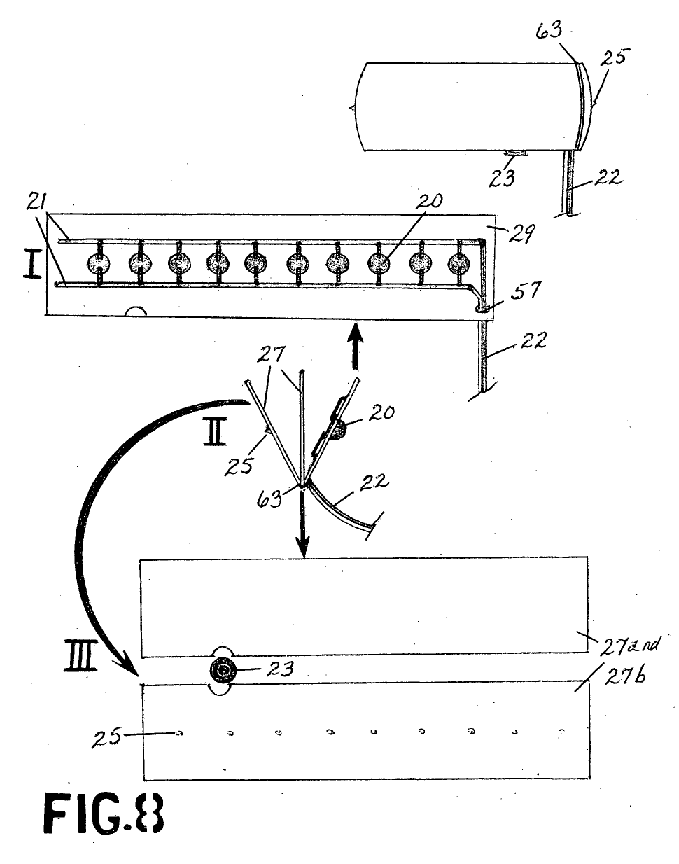

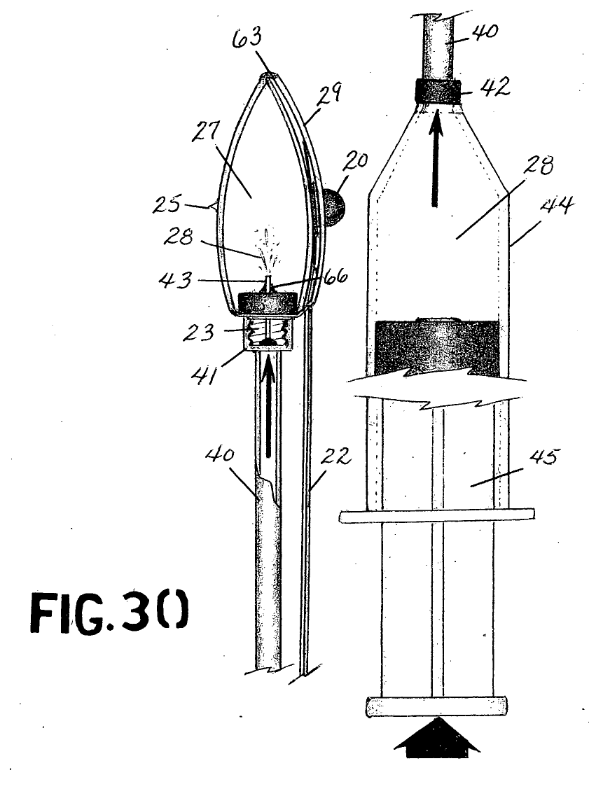

FIG. 8 shows two more separate layers of plastic 27a and 27b, that will eventually be joined to create a saline 28 containment area 27, as shown in FIGS. 30 and 43.

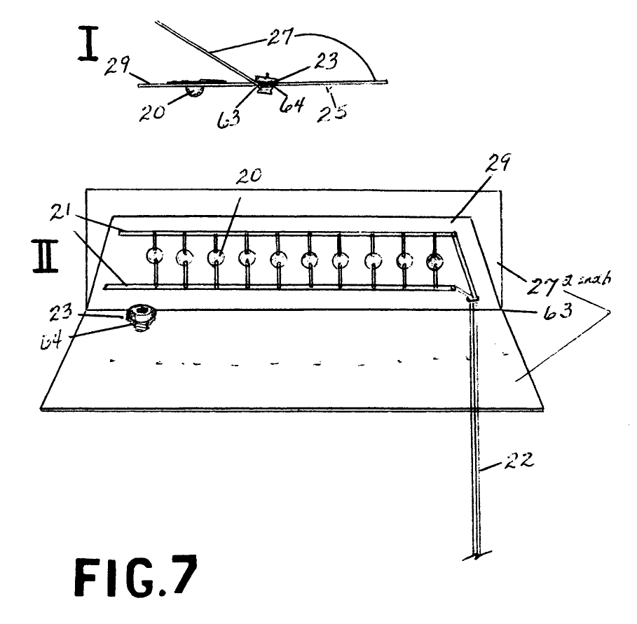

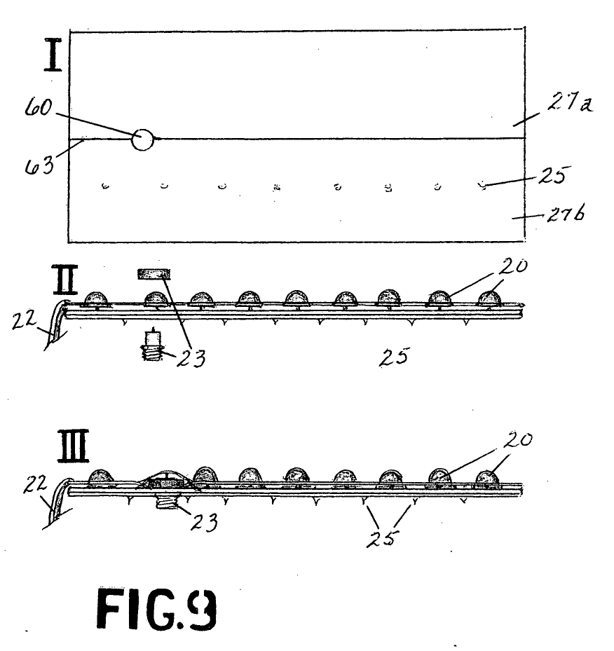

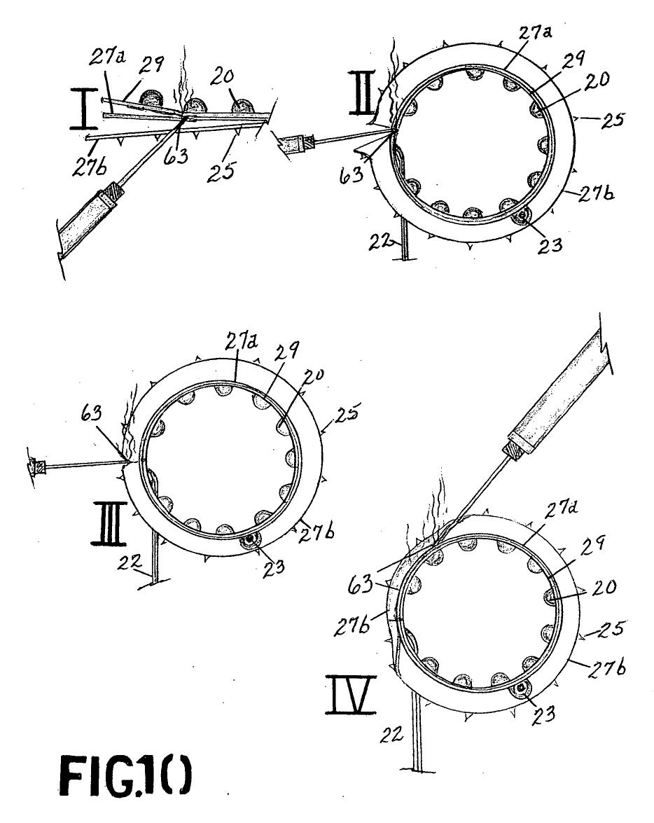

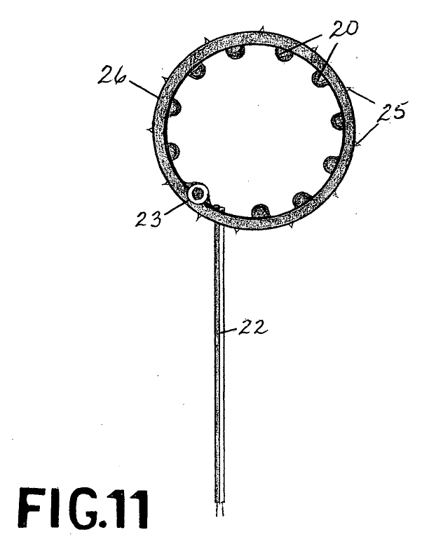

Bottom ends of the three layers of plastic 27a, 27b and 29 are heat-sealed 63 at their bottom edges as shown in FIGS. 7 and 10, with burrs 25 facing outward on the circle's 26 surface, and positioned before heat-sealing 63 to where the valve 23 insertion hole 60 remains in line, as shown in FIG. 9-I, and FIG. 7-II. FIG. 10 shows several actual heat-sealing 63 processes.

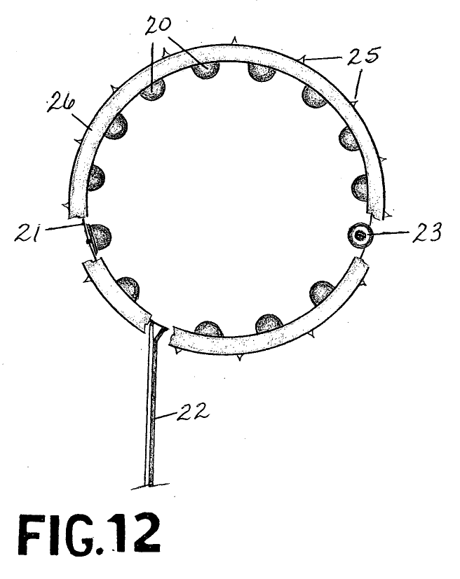

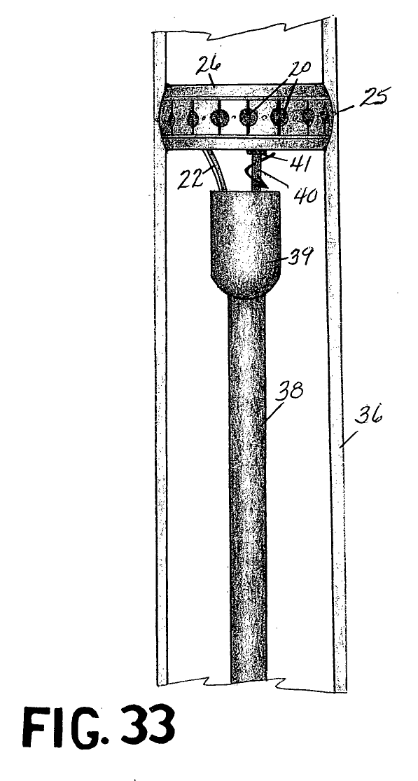

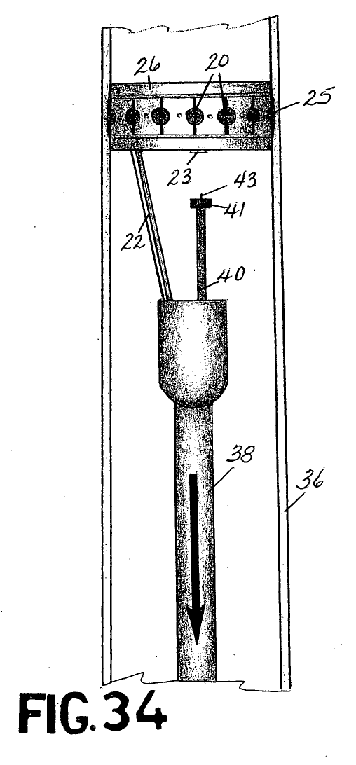

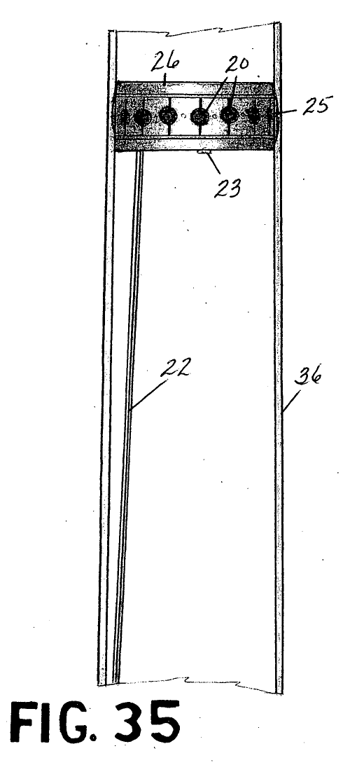

Soft plastic layer 27b, which will ultimately become the outwardly-facing surface of the circle 26, has multiple burrs 25 running lengthwise and at the center of its surface, as shown in FIGS. 11 and 12. These plastic burrs 25 will provide stationary stability for the circle 26 of LEDs 20, once it has been inflated in a vein 36, as shown in FIGS. 33 and 34.

With soft plastic layers 27a, 27b and 29 heat-sealed along their bottom edges, the valve 23 hole 60 will be perfectly round and sized to afford a snug, airtight fit when the valve 23 is pressed into the hole 60 from beneath. A coating of adhesive sealer 64 precedes placement of the valve's 23 securing ring which holds the valve 23 in position, and creates an airtight, leak-proof seal, as shown in FIG. 9-I, II and III, and FIG. 7-I and II.

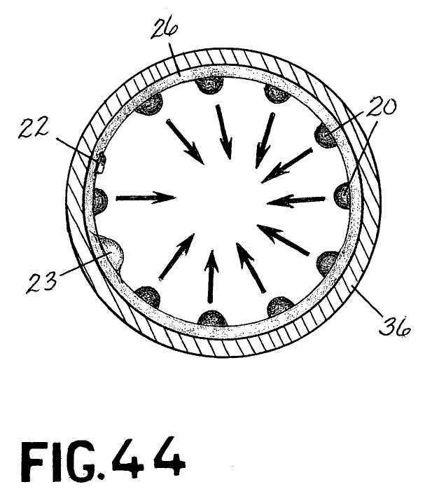

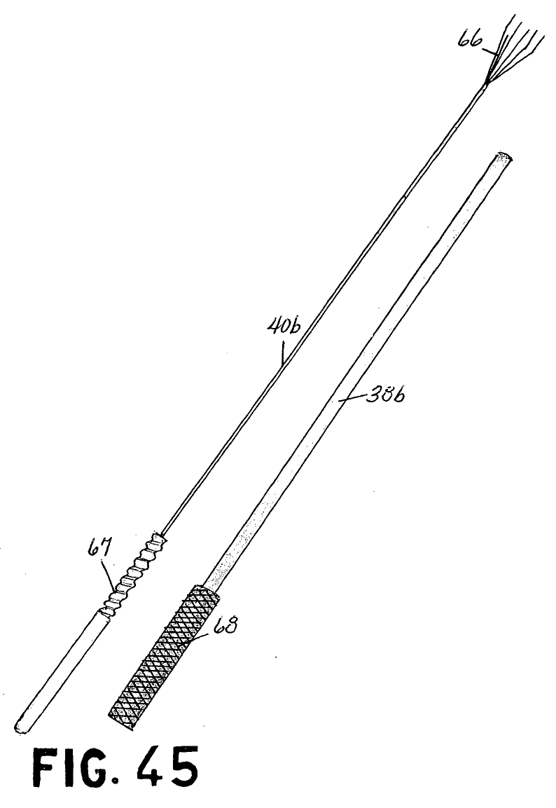

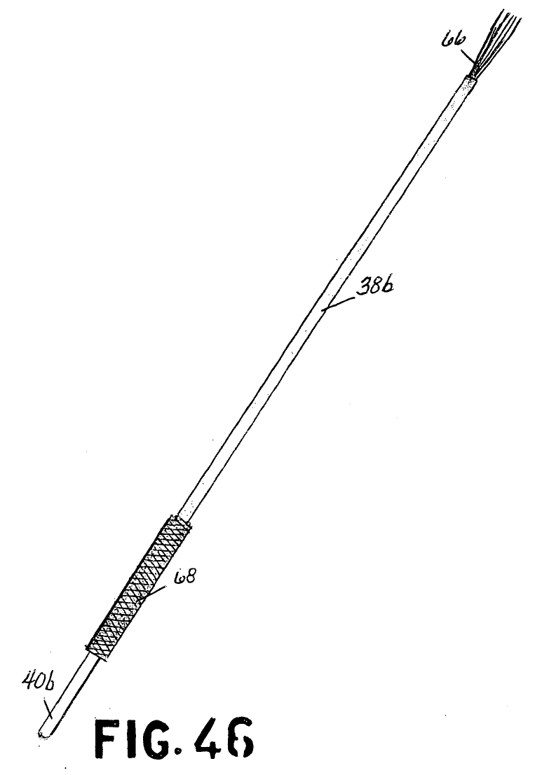

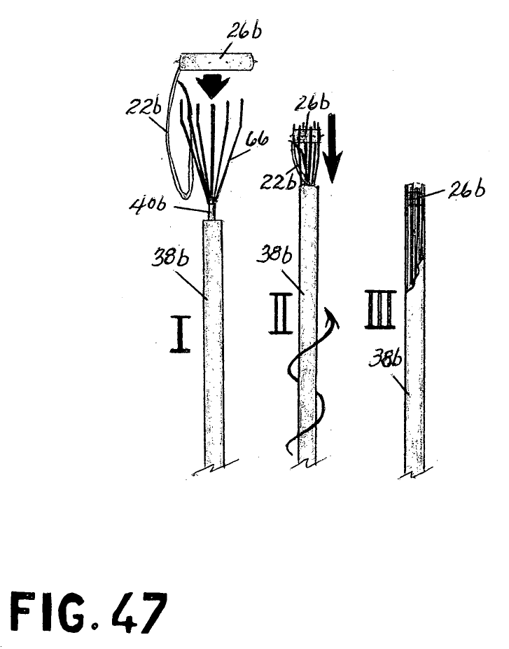

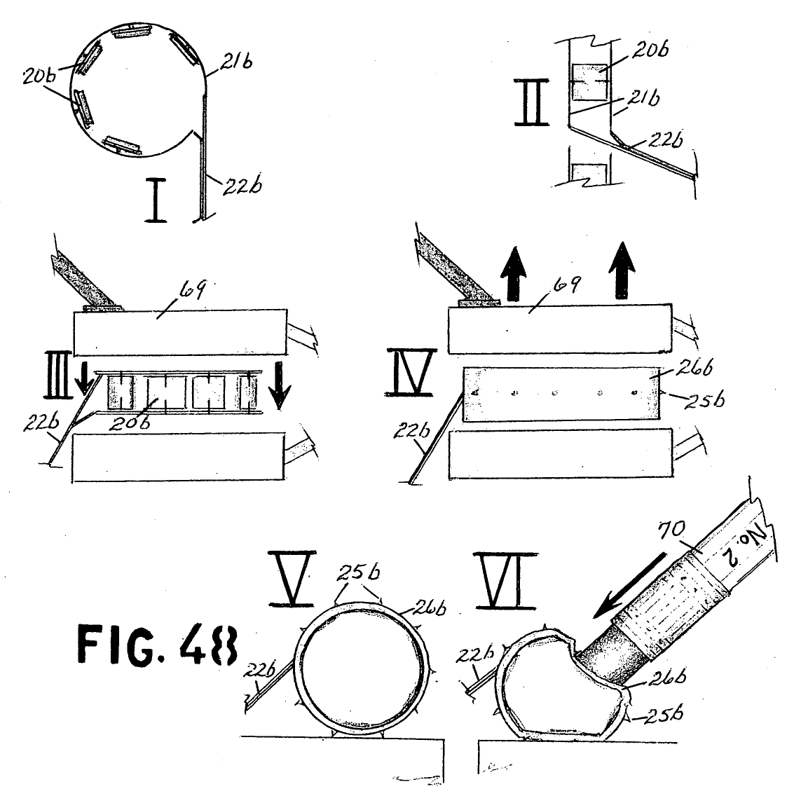

With valve 23 positioned, the top edges of soft plastic layers 27a and 29 are the heat-sealed 63, as shown in FIG. 10-I. Once heat-sealing 63 is affected, both ends of all three layers are folded until opposite ends meet, as shown in FIG. 10-II. The ends of layers 27a and 29 are then heat-sealed 63 together to create a circle 26, with LEDs 20 facing inwardly toward the center of the circle 26, as shown in FIG. 10-II, FIG. 11, and FIG. 44.

The outside layer 27b, with outwardly-facing burrs 25, has a slightly longer length than layers 27a and 29. Separate ends of layer 27b are heat-sealed 63 together as shown in FIG. 10-III, creating a constantly running space between layers 27a and 27b. This “space” will become the saline 28 containment area 27 once the edges of all three layers are heat-sealed.

With all bottom and end edges now heat-sealed 63, the top edges of layers 29 and 27a are then sealed 63, creating an airtight enclosure for tracks 21 and LED 20 and cord 22 contact points. At this point the top edge of layer 27b is carefully heat-sealed 63 along the heat-sealed 63 seam at the top of layers 27a and 29.

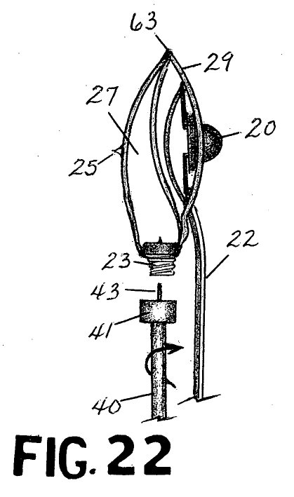

FIGS. 22 and 30 show section views where heat-sealing 63 has been affected to create what has now become the saline 28 containment area 27. It is into this area 27 or space that saline 28 will be injected through the one-way valve 23 as it passes through the feed tube 40 under pressure from pressing the plunger 45, forcing saline 28 from the syringe. Circle 26 of LEDs 20 is now assembled.

At the opposite end of the two-strand cord 22, as shown in FIG. 2, is a circular hard plastic circuit containment compartment 31. This compartment 31 contains two contact screws 48, a battery 32 and battery bracket 50, a signal receiver 47 for a remote-control 46, and an assortment of resistors 55. Combined, this circuit provides a constant, non-fluxuating energy source which activates the multiple LEDs 20 within the light-radiating circle 26 at the opposite end of the two-strand cord 22, when the “ON” button 53 of the remote-control 46 is pressed.

Utilizing invention 100 as an intravenous light “filter” to destroy the replicative values of viruses or cancerous cells requires a procedure of surgery that necessitates the use of a syringe 44, a catheter 38 with capsule 39 end, a saline 28 feed tube 40 with threaded end 42 that corresponds with the threaded mouth 65 of the syringe 44, and a threaded cap 41 at the feed tube's 40 opposite end with a blunt, hollow needle 45 at its center designed to be threaded onto and inserted into the LED 20 circle's 26 one-way valve 23. A container of saline 28 is required, as is an air bleed-valve 24 that allows for a total collapse of the saline 28 containment area 27, so the rolling and folding of the LED 20 circle 26 into a small enough “package” to fit down into the capsule 39 end of the catheter 38 can be adequately affected before intravenous insertion may begin. The circuit containment compartment 31 is the last component of the procedure.

To begin the assembly of insertion components, the threaded end 42 of the saline 28 feed tube 40 is threaded down onto the threaded mouth 65 of the syringe 44, as shown in FIG. 16.

FIG. 17 shows the end of the feed tube 40, submerged into a bottle of saline 28. As plunger 45 of the syringe 44 is pulled down, as indicated by arrow, saline 28 is drawn through the blunt needle 43, through the feed tube 40, and into the syringe 44.

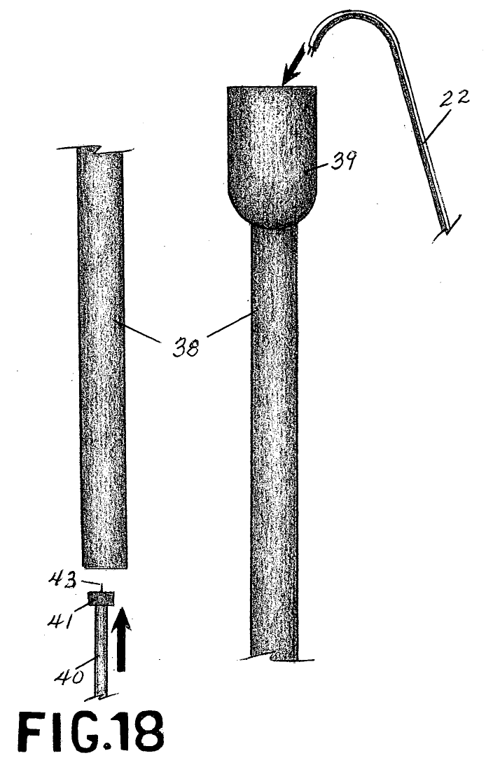

Once syringe 44 is filled with saline 28, the insertion catheter 38 must be prepared. As shown in FIG. 18, the two-strand cord 22 is fed down into the capsule 39 end of the catheter. When cord 22 has protruded from the opposite end of the catheter 38, the end of the feed tube 40 with blunt needle 43 at its center is fed into the bottom end of the catheter 38 while cord 22 is held in place.

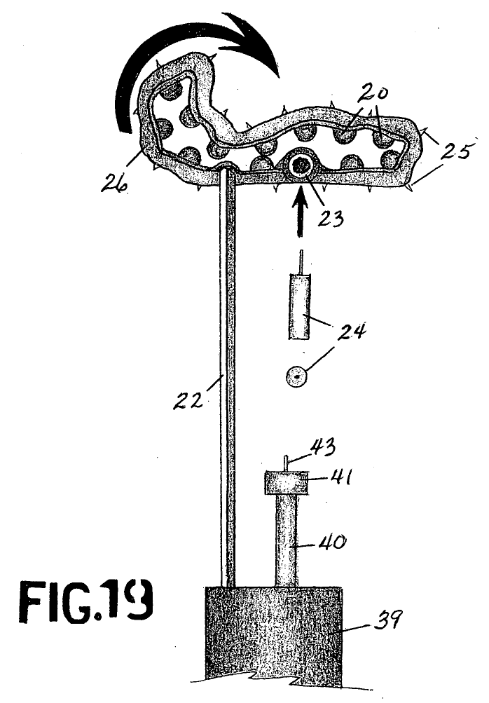

FIG. 19 shows cord 22 and feed tube positioned inside catheter 38. Air bleed-valve 24 must now be inserted in saline 28 conduit valve 23 to allow trapped air to evacuate from the saline 28 containment area 27 of the circle 26 of LEDs to allow “package” to be rolled, as larger curved arrow indicates, into its most compact state.

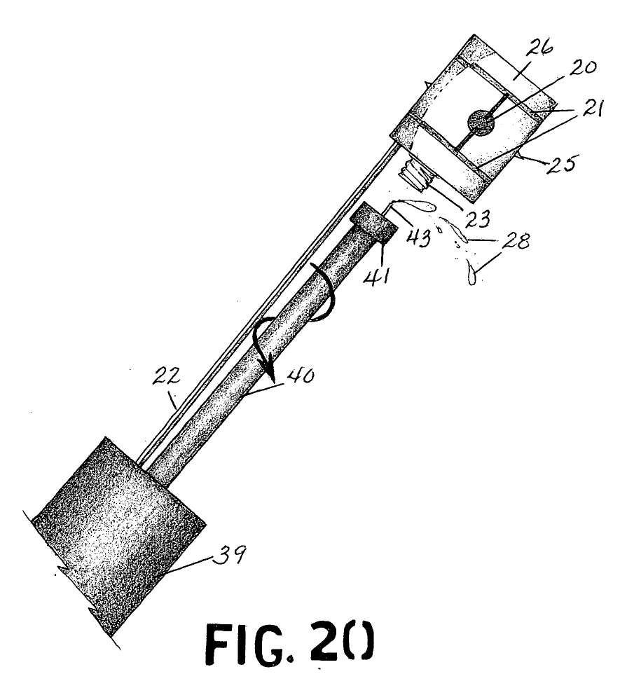

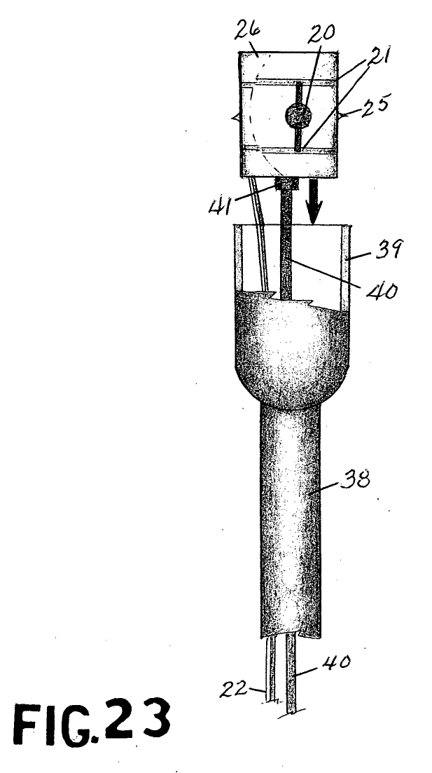

As shown in FIG. 20, once the circle 26 of LEDs 20 has been affectively rolled into its most compact state, plunger 45 of syringe 44 is pressed to insure feed tube 40 is filled to the tip of its blunt needle 43 with saline 28. When verification of filled feed tube 40 is complete, threaded end 41 and blunt needle 43 are aligned with the threaded valve's 21 mouth. The syringe 44 at the opposite end of the catheter 40, as seen in FIG. 32, is turned clockwise as indicated by spiraling arrow in FIGS. 20, 21 and 22. FIG. 23 shows threaded end 41 firmly attached to the valve 23. Arrow indicates “package” is ready to be pulled down into the catheter's 38 capsule 39 by-way of pulling the syringe 44 at the opposite end of the catheter 38.

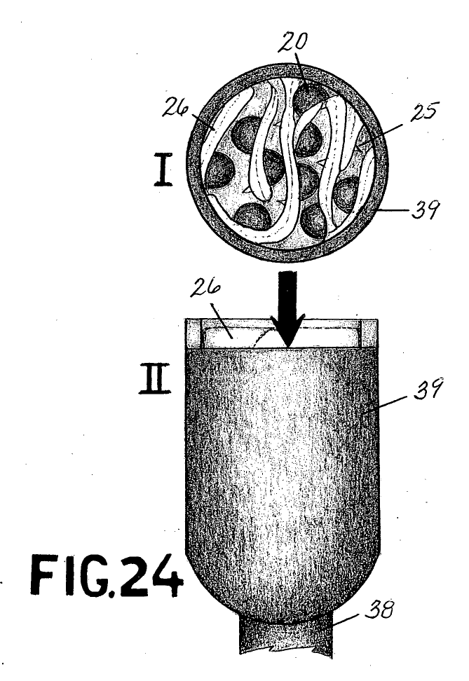

FIG. 24-I shows an aerial view of “package” recessed down into the capsule 39. FIG. 24-II shows a partial section view of the top edge of the folded circle 26 of LEDs 20, now ready for insertion into a recipient's thigh 34 incision 35, and on into the selected vein 36.

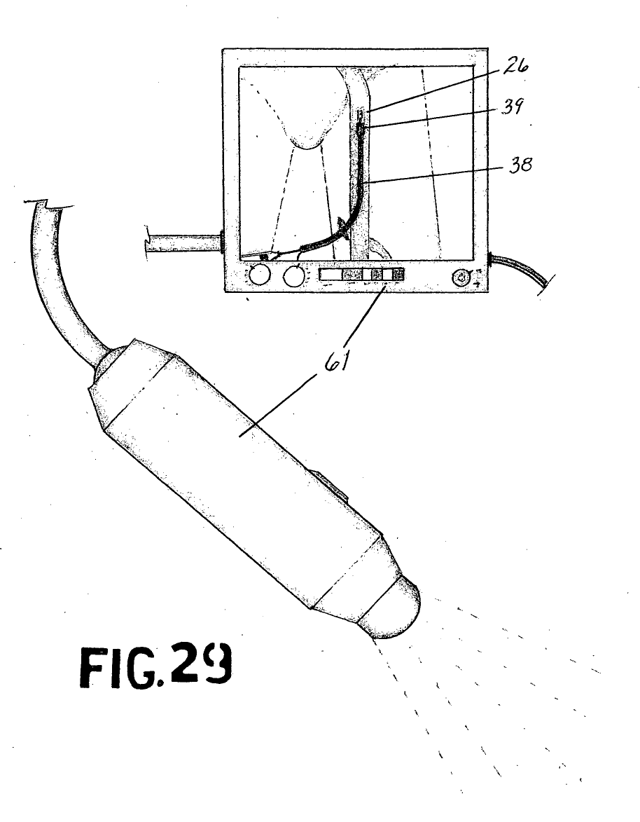

It should be noted that the insertion process utilizes a method of tracking the depth, direction and eventual deployment of the circle 26 of LEDs 20 inside the selected vein 36, called ultrasonography. An ultrasound device 61, as shown in FIG. 29 relays images to a monitor which allows for an accurate, precise positioning and placement of the intravenous implant.

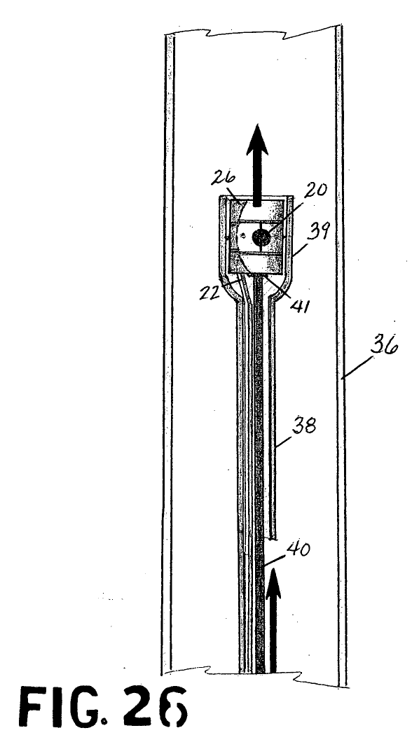

FIG. 26 shows a section view of the catheter 38, its capsule 39, and the “package”, cord 22 and feed tube 40, in position in the recipient's common iliac vein 36. Arrows indicate direction of movement up into the vein 36.

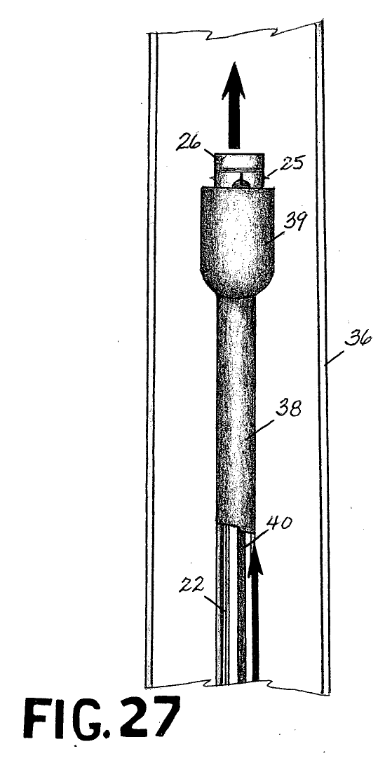

FIG. 27 shows “package” exiting capsule 39 as syringe 44 at the opposite end is pressed into the catheter 38.

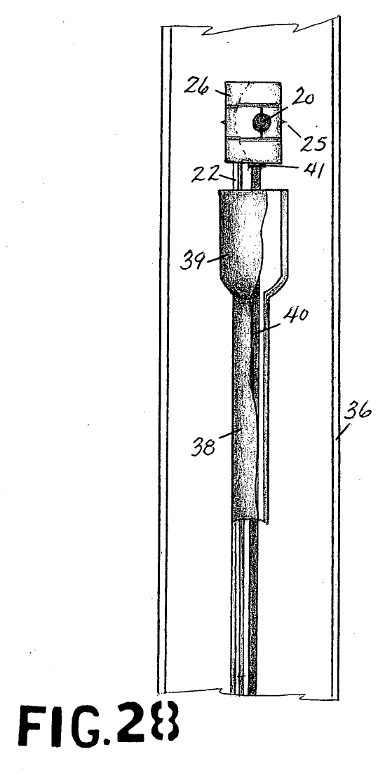

FIG. 28 shows “package” now fully in view as shown on the monitor in FIG. 29. The process of inflating the saline 28 containment areas 27 may now proceed.

FIG. 30 shows cut-away views of saline 28 leaving the open end of the blunt needle 43, filling the saline 28 containment area 27, as the plunger 45 is pressed, as the wide arrow indicates, forcing saline 28 from the syringe 44, as the narrow arrow indicates, through the saline 28 feed tube 40, and into the containment area 27.

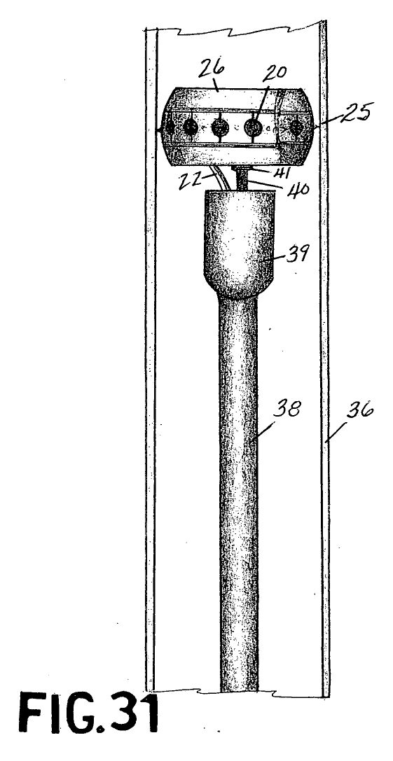

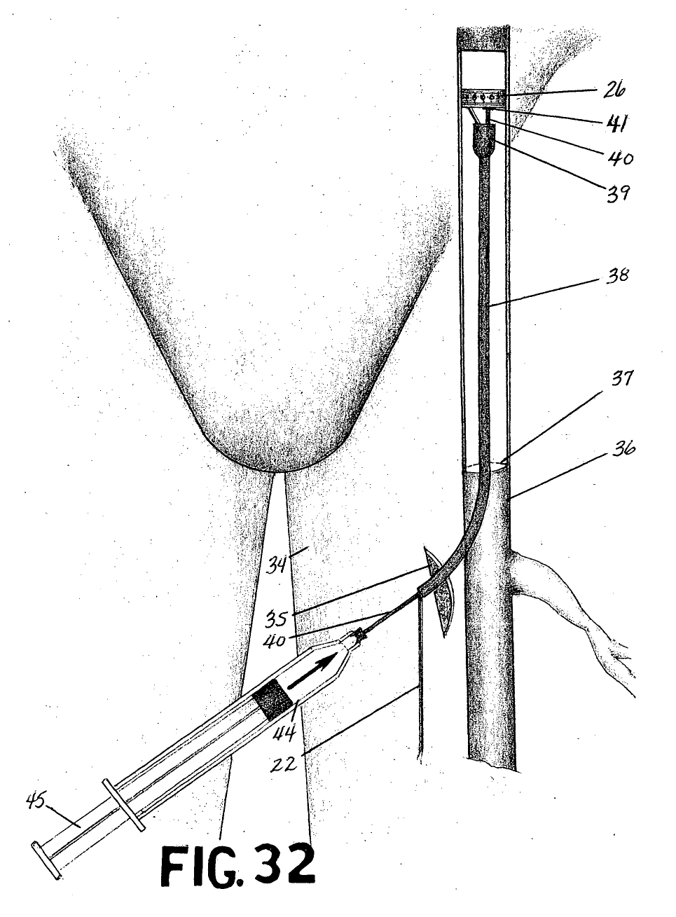

FIG. 31 shows the circle 26 of LEDs 20 inflating and taking on its circular shape. Note the burrs 25 encircling the center of the outer surface of the circle 26.

FIG. 32 shows the entire assembly of syringe 44, feed tube 40, and catheter 38. Circle 26 of LEDs 20 is fully inflated. Arrow again indicates plunger 45 pressing saline 28 into the containment area 27.

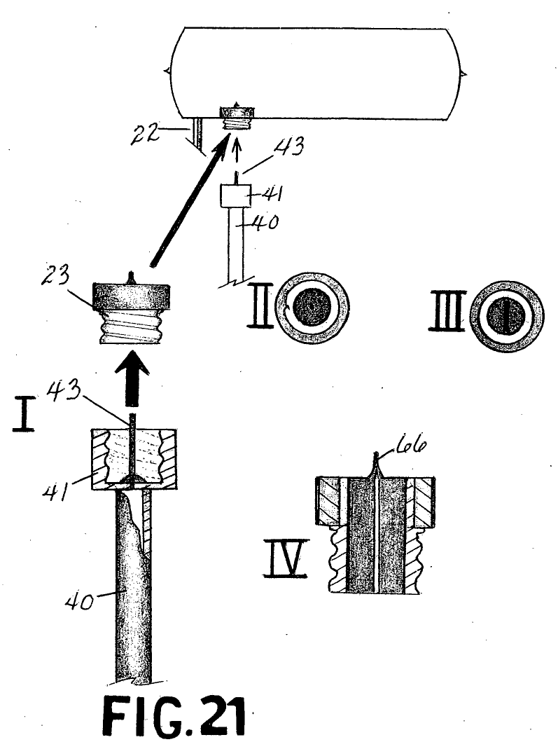

FIG. 33 shows circle 26 fully inflated. Burrs 25 have now indented into the inner-surface of the vein 36 where they will serve to cause the circle 26 to remain stable and stationary. The feed tube 40 is now disconnected from the valve 23 by turning syringe 44 at the opposite end of the catheter 38. As feed tube 40 is removed, tiny rubber flaps 66, as shown in FIG. 21-IV, are pressed closed by the pressure of the saline 28 entrapped within the containment area 27, serving to “trap” saline inside the area 27.

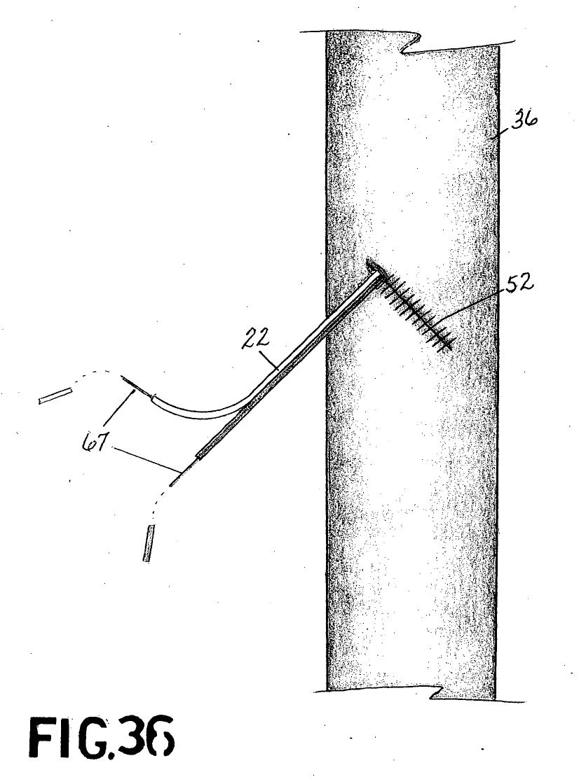

FIG. 34 shows feed tube 40 disconnected from the valve 23. Catheter 38 and feed tube 40 are now pulled down and extracted from recipients vein 36, then out through the incision 35 on the inner-thigh 34. Incision 37 in the common iliac vein is now sutured 52 or “lased” tightly around protruding cord 22, as shown in FIG. 36. Insulation is trimmed from the end of both strands, after strands are separated, to expose copper wire 67.

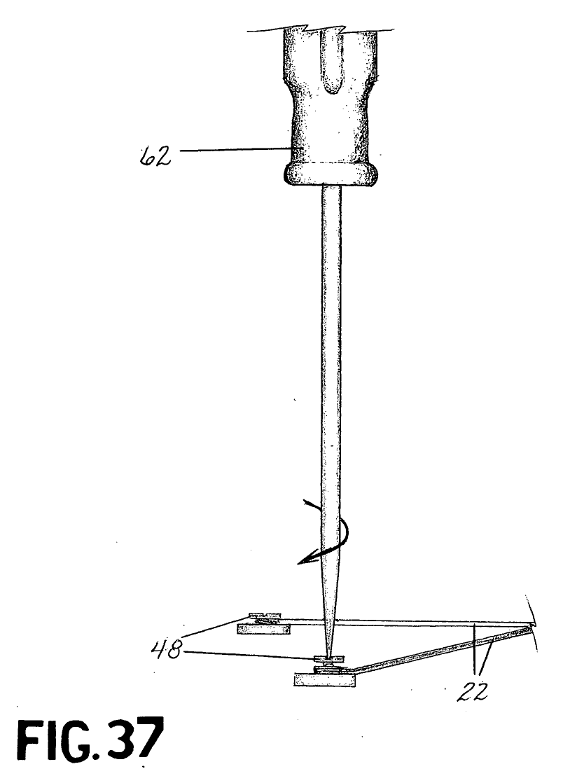

FIG. 37 shows copper wire 67 at the ends of the separated strands of the cord 22 as they are securely attached to contact points 48. Screws In contacts 48 are tightened with a screw driver 62, as arrow indicates.

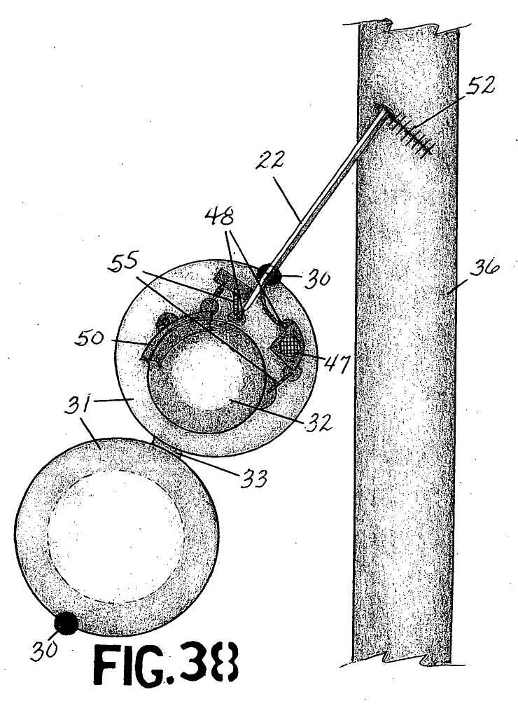

FIG. 38 shows both strands of the cord 22 attached to the contacts 48, completing the circuit housed within the containment compartment 31. The compartment 31 may now be closed at its hinge 33. Soft rubber seals 30 seal area around the cord 22 where it exits the compartment 31. Compartment 31 is sealed shut with non-toxic adhesive and made ready for concealment beneath the skin of the inner-thigh 34.

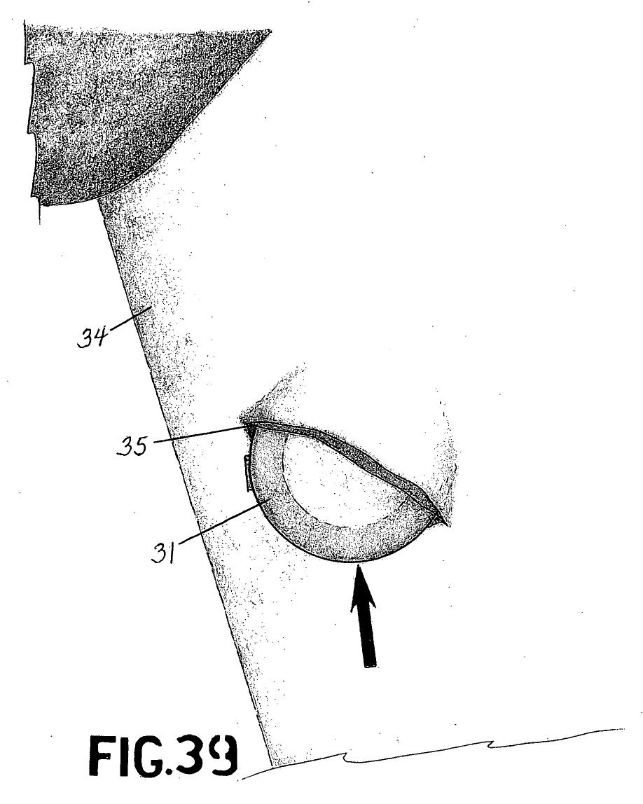

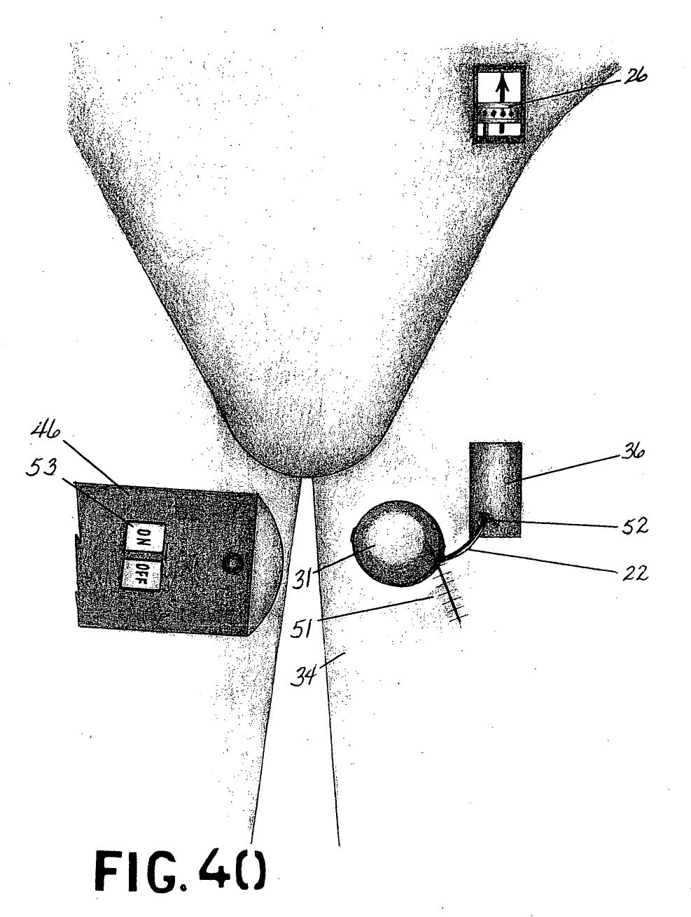

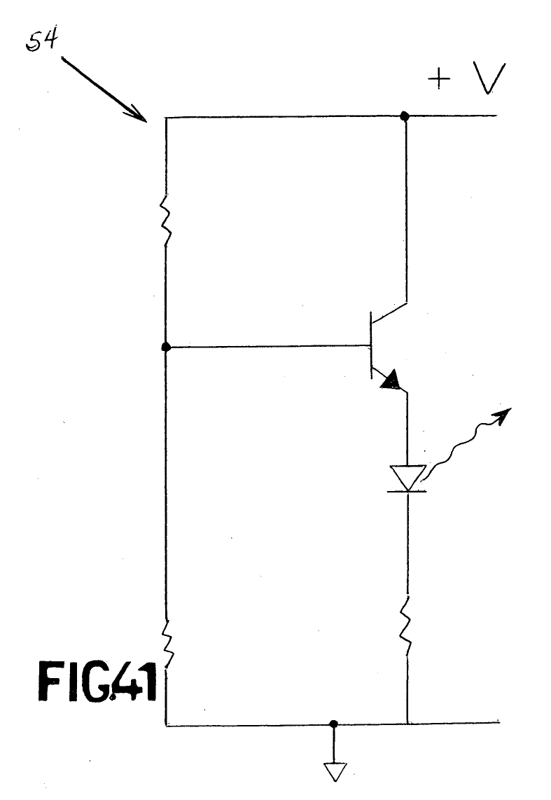

FIG. 39 shows containment compartment 31 as it is being inserted through the incision 35 on the inner-thigh 34.

With compartment 31 insertion process complete, and incision 35 on the inner-thigh 34 is sutured 51 closed, the “ON” button 53 on the remote-control 46 is pressed to activate the radially-positioned LEDs 20 that line the inside of the circle 26. Arrow in cut-away view shows the direction of blood-flow through the circle's 26 light. Circle 26, cord 22 and containment compartment 31 are the “invention” 100 that remains implanted.

FIG. 41 shows a schematic 54 of a constant power circuit, designed to safeguard the LEDs 20 from an increased electrical energy that could accidentally increase intensity of laser or non-laser light radiated from the selected LEDs 20 to combat a specific targeted virus or cancerous infestation.

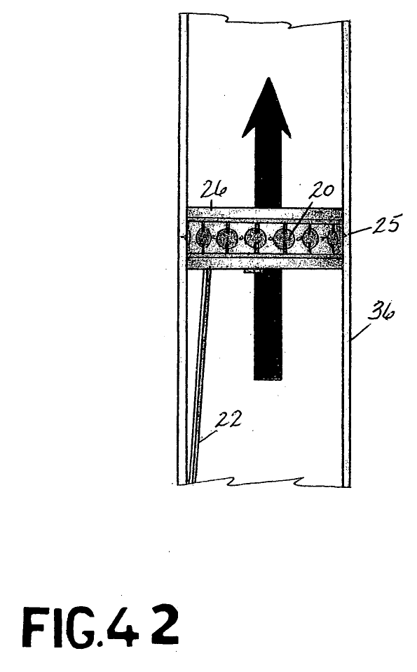

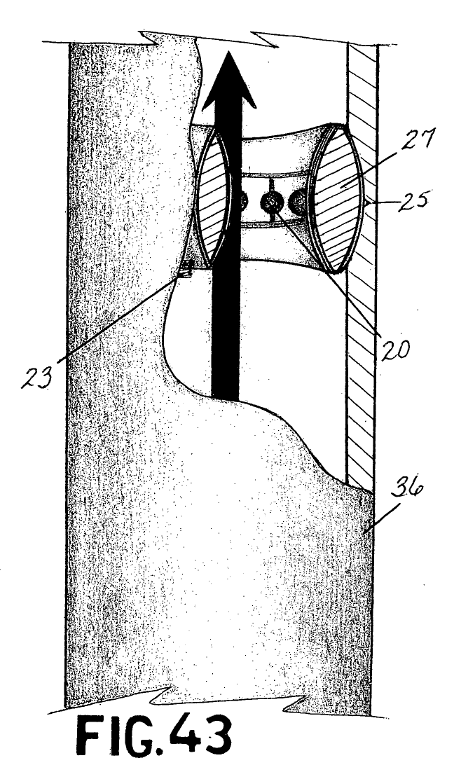

FIG. 42 shows activated LEDs 20. Arrow indicates the direction of circulated blood.

FIG. 43 shows a section view of the circle of LEDs 20 and its saline 28 containment area 27. Arrow indicates the direction of circulated blood.

FIG. 44 shows an aerial view of a cross-section of the vein 36, looking down into the circle 26 of LEDs 20. Arrows indicate the direction of irradiated laser or non-laser light, radiating into circulating blood, from total-surface light-emitting diodes 20.

### Alternative Embodiments

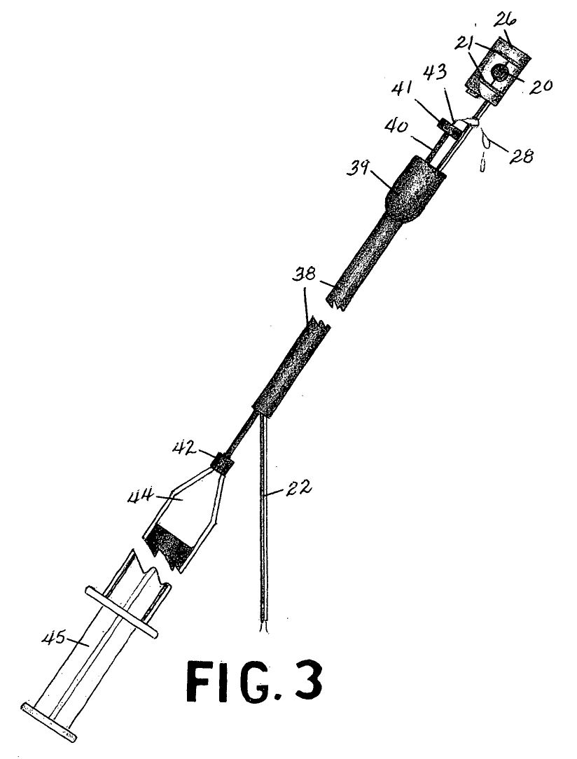

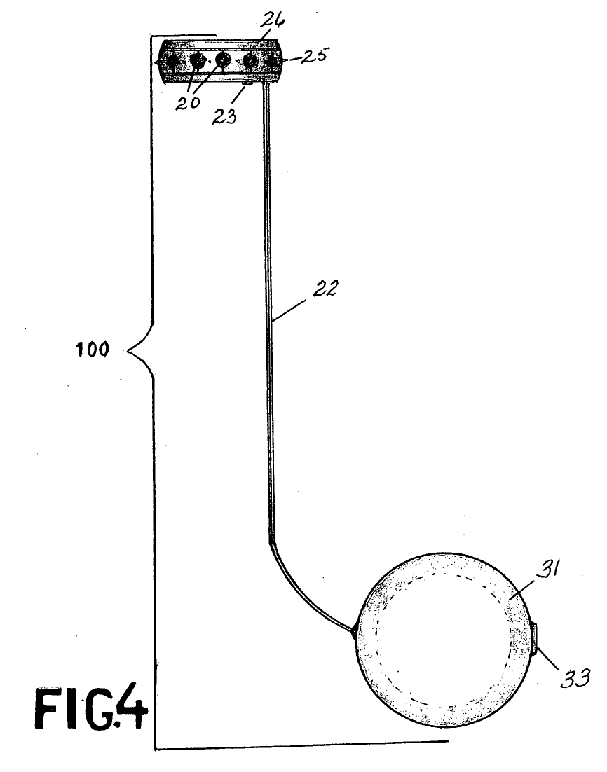

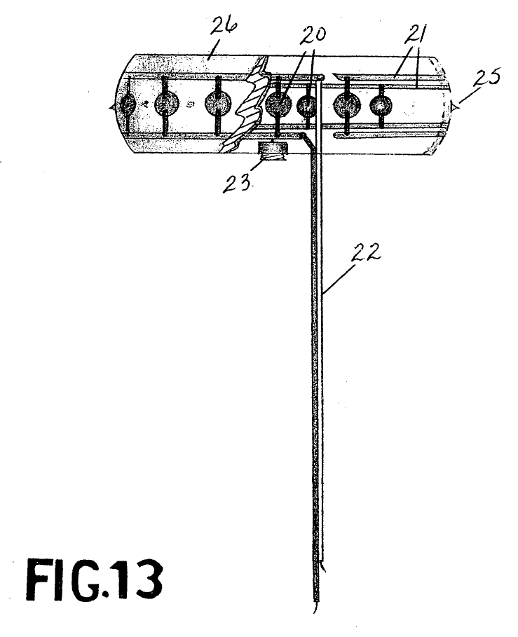

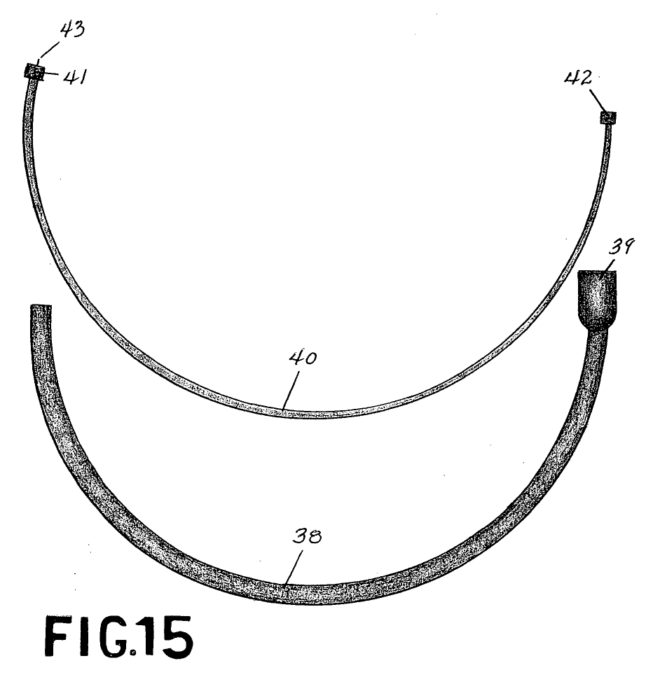

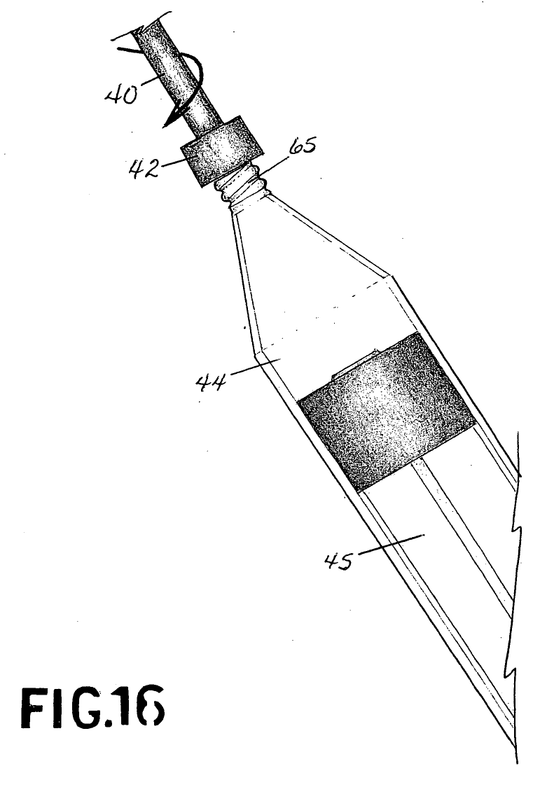

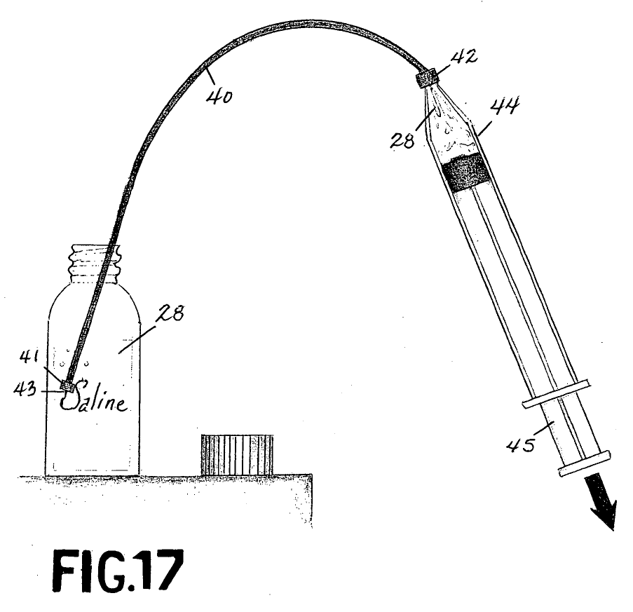

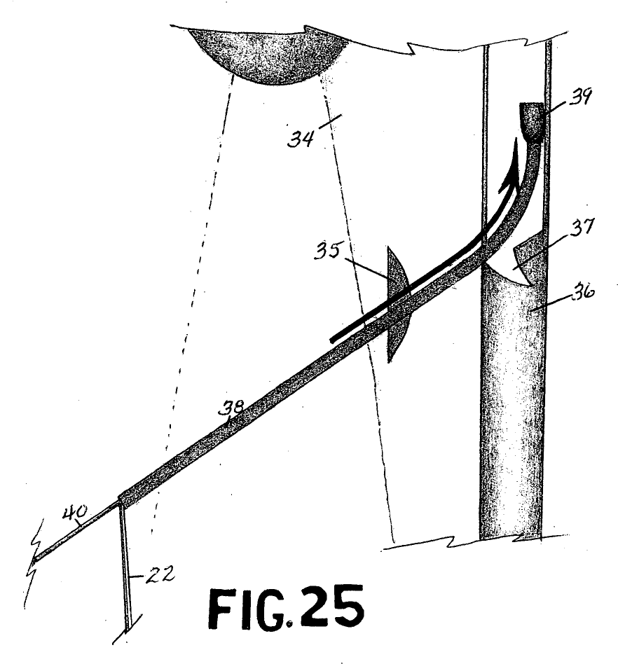

As stated on page 14 of this application under the heading of “Detailed Description of Preferred Embodiments”, in paragraph one, specific details in FIGS. 1-44 are not to be interpreted as limiting, but rather as a basis for the claims.

The following FIGS. 45-48 are intended to establish an alternative design where light-emitting diodes and contact tracks are housed within an injector molded body, fabricated of a non-toxic, flexible, collapsible material, such as nylon. Flat-faced LEDs replace conventional round-faced ones intended to alleviate the necessity of blood thinning agents used to reduce coagulation.

Primary claims regarding photodynamics, phototherapy and utilization of psoralens and fetosecond laser light vibrative energy remain as on pages 24, 25, and 26 herein.

### Brief Description of FIG. 45-48

FIG. 45 Shows two components that comprise insertion implements to affect deployment of the molded nylon body encasing flat-faced LEDs and contact tracks.

FIG. 46 Shows the two components comprising insertion implements, assembled.

FIG. 47 Shows a 3-step process of inserting the collapsible, nylon-housed circle of LEDs in preparation for deployment into a human blood vessel.

FIG. 48 Shows I, an aerial view of flat-faced LEDs, contact tracks and cord; II, an underside view of a flat-faced LED attached to contact tracks with cord; III, an assembled circle of LEDs, attached to tracks and cord, positioned for injector molding; IV, injector molding process completed leaving LEDs, tracks and cord contacts housed within a sealed, nylon body; V, a view looking down into the nylon encased circle of LEDs, tracks and cord, and VI, a common No. 2 pencil pressing the collapsible circle to show the circle's flexibility. Pencil provides perspective of approximate circumference of circle.

### Numbered Components of FIGS. 45-48

20b: Flat-faced LEDs
21b: Contact tracks
25b: Burs
26b: Nylon circle
38b: Catheter
40b: Guide rod
66: Retracting metal
68: Textured grip, with inner-threading to correspond with threaded guide rod
69: Plastic injector mold
70: No. 2 pencil

What is claimed is:

1. Intravenous laser/non-laser light-emitting diode implant for destroying blood borne viral infestations and other malign cells, integrated among blood components in a human circulatory system comprised of:
a plurality of select laser or non-laser, total surface light-emitting diodes (LEDs), attached radially in an inwardly-facing manner to a flexible, inflatable ring; a one-way injection valve for inflation/sealing of ring;
a two-track contact points; a power cord attached at one end to both contact points, and at the other end to contact points with a circuit containment compartment; a containment compartment that houses a battery power source, signal receiver for activation/deactivation, as well as other standard electronic components such as resistors and battery contacts.

2. Intravenous laser/non-laser light-emitting diode implant for destroying blood borne viral infestations and other malign cells, integrated among blood components in a human circulatory system as claimed in claim 1, wherein the inflatable ring of LEDs is installed by catheter within a major vein to subject malign cells incorporated among blood components passing through said ring of LEDs to be irradiated by a select nanometers of laser or non-laser light.

3. Intravenous laser/non-laser light-emitting diode implant for destroying blood borne viral infestations and other malign cells, integrated among blood components in a human circulatory system as claimed in claim 1, wherein said total-surface LEDs are utilized in the process of irradiating targeted malign cells may be of any of a multitude of specific colors of laser or non-laser LEDs as required in applications of a specific nanometer to destroy a targeted infestation of cancerous blood cells or viruses.

4. Intravenous laser/non-laser light-emitting diode implant for destroying blood borne viral infestation and other malign cells, integrated among blood components in a human circulatory system as claimed in claim 1, wherein the necessity of introducing light-sensitizing chemicals into the subject blood to create vulnerability of malign cells to light radiated from the intravenously implanted circle of LEDs through which malign cells must pass in their course through the circulatory system, incorporated with circulating blood.

5. Intravenous laser/non-laser light-emitting diode implant for destroying blood borne viral infestations and other malign cells, integrated among blood components in a human circulatory system as claimed in claim 1, wherein a person ingests psoralens in the synthetic form of 8-methoxypsoralen, thereby introducing psoralens into the person's blood where it intermingles with said person's blood and, as psoralen-laced blood circulates through a circle of ultraviolet A producing total-surface LEDs, where psoralens irradiated by this light become active molecular surgeons, microscopically chipping away the chemical bonds that hold together the deoxyribonucleic acid (DNA) of the cells involved in lymphomatic leukemia and T-cell lymphoma.

6. Intravenous laser/non-laser light-emitting diode implant for destroying blood borne viral infestations and other malign cells, integrated among blood components in a human circulatory system as claimed in claim 1, wherein the utilization of low-power pulsating laser light-emitting diodes operating the purple portion of the visible color spectrum at the wavelength of 425 nanometers, at a pulse width of one hundred quadrillianths of a second (100 femtoseconds), which instantly affects the bursting of a viral cell's protein shell (capsid), destroying this viral envelope and rendering the virus incapable of accessing a “host” lymphocyte blood cell where the virus would have otherwise commandeered the host cell's DNA to replicate itself, thus diminishing the viral load in the blood of the invention's recipient.
